(19) United States
(12) Patent Application Publication
(10) Pub. No.: US 2022/0081670 A1
(43) Pub. Date: Mar. 17, 2022

(54) Incubator and Shelf with Integrated Microscope and Wireless Transmitter

(71) Applicant: Anthony Walter Demsia, Sayreville, NJ (US)

(72) Inventor: Anthony Walter Demsia, Sayreville, NJ (US)

(21) Appl. No.: 17/018,341

(22) Filed: Sep. 11, 2020

(51) Int. Cl. C12M 1/00; G02B 21/24; G02B 21/36; G02B 21/06; G02B 21/28

(52) U.S. Cl. CPC C12M 41/14; G02B 21/242; C12M 41/08; G02B 21/06; G02B 21/28; G02B 21/36

(57) ABSTRACT

An incubator and shelf with integrated microscope and wireless transmitter comprises a shelf adapted for use inside an incubator; a microscope integrated into the shelf; and a wireless (such as Wi-Fi) transmitter that wirelessly transmits an image produced by the microscope. Embodiments may be powered by the incubator, and may have an inverted microscope that views an object from below.

### BACKGROUND OF THE INVENTION

The present invention generally relates to incubators for growing cells and more specifically to an incubator having a shelf with an integrated microscope and a wireless transmitter.

Carbon dioxide (CO2) incubators are used in various forms of research for growing cells. It is common practice to remove the cells from the incubator periodically, perhaps daily, to place them under a microscope for viewing. This requires removing the cells from their optimal growth environment (the incubator with controlled temperature and CO2 percentage) risking damage to the cells. This also requires opening the incubator door, and exposing the cells and incubator chamber to potential contaminants

It would be desirable to have an incubator and a shelf for incubators with an integrated microscope, with a wireless transmitter so that the object of the microscope can be viewed without opening the door to the incubator.

### SUMMARY OF THE INVENTION

In one aspect of the present invention, a device comprises a shelf adapted for use inside an incubator; a microscope integrated into the shelf; and a wireless transmitter that wirelessly transmits an image produced by the microscope.

In another aspect of the present invention, a device for use with an incubator and a display outside the incubator, comprises a shelf adapted for use inside the incubator and adapted to receive power from the incubator; a microscope integrated into the shelf that produces a magnified image of an object inside the incubataor; and a wireless transmitter that wirelessly transmits the magnified image to the display.

In yet another aspect of the present invention, a device comprises an incubator; a display outside the incubator; a shelf adapted for use inside the incubator and adapted to receive power from the incubator; a microscope integrated into the shelf that produces a magnified image of an object inside the incubataor; and a wireless transmitter that wirelessly transmits the magnified image to the display.

### BRIEF DESCRIPTION OF THE DRAWINGS

FIG. 1 depicts a front view of an embodiment of a microscope according to the present invention;

FIG. 2 depicts a rear view of the embodiment of FIG. 1;

FIG. 3 depicts an embodiment of a microscope mounted on a shelf according to the present invention;

FIGS. 4A and 4B depict top views of microscopes mounted on shelves according to the present invention; and

FIGS. 5A, 5B, and 5C depict an embodiment of a light mask according to the present invention.

### DETAILED DESCRIPTION

The preferred embodiment and other embodiments, which can be used in industry and include the best mode now known of carrying out the invention, are hereby described in detail with reference to the drawings. Further embodiments, features and advantages will become apparent from the ensuing description, or may be learned without undue experimentation. The figures are not necessarily drawn to scale, except where otherwise indicated. The following description of embodiments, even if phrased in terms of “the invention” or what the embodiment “is,” is not to be taken in a limiting sense, but describes the manner and process of making and using the invention. The coverage of this patent will be described in the claims. The order in which steps are listed in the claims does not necessarily indicate that the steps must be performed in that order.

An embodiment of the present invention generally provides an incubator and a shelf for an incubator that has an integrated and mounted microscope. Embodiments of the microscope may use wireless transmission, such as a Wi-Fi, to project the signal to the display on the front door. Embodiments may allow the user to project to a smart phone or Wi-Fi device.

Embodiments of a microscope may be integrated into the shelf of an incubator and powered from the incubator. The incubator may include a display that allows viewing of the cells. The display may be integrated with, and powered by, the incubator.

Embodiments of an incubator may have controls on the outside to operate the microscope. This could include fine focus, with a gross focus control such as a dial on the microscope itself. Embodiments of a microscope may use Wi-Fi to project the signal wirelessly. That way, a user can view the object of the microscope, such as a cell culture dish, flask, well plates, etc., without needing to open the door to the incubator oven.

Embodiments of a microscope may include a light holder attached to a top mast, which raises and lowers the light holder relative to a stage plate. The light holder and stage plate may be made of white acrylic. A light mask slider may slide onto the light holder in either of two orientations, which allows for a different illumination conditions. The top mast and lower mast may be made of grey anodized aluminum.

Embodiments of an inverted microscope may have a window in a stage plate that allows a microscope lens below the object to view the object.

Embodiments may include a printed circuit board (PCB) and a battery in a housing. The PCB housing may be grey anodized aluminum. A space between the PCB housing and the stage plate may allow for heat from the shelf to dissipate.

In embodiments, a light mask slider may be installed in a first orientation (upside down), with a window to let light through, down to the object and the microscope lens. A light mask slider may be installed in a second orientation which has a deflector, which only lets light out the sides.

In embodiments, a Wi-Fi or other wireless transmitter with PCBs or other chips generate heat, which may warm the microscope. To help maintain constant temperatures inside the incubator, the microscope and other components may be thermally protected or separated by a distance from the object to be microscopically viewed. This may include a plate of cell samples, resting on the stage plate of the microscope.

Embodiments may include a coarse adjustment control, such as a knob, on the microscope. An objective lens or lenses may produce an image on a photo sensor, which is wirelessly, digitally transmitted outside of the incubator to a display.

Embodiments of an incubator have interior shelves that are thermally controlled, and outside surfaces, such as the front of the door.

In embodiments, indicator LEDs on the stage plate may indicate the status of the microscope (e.g. on or off).

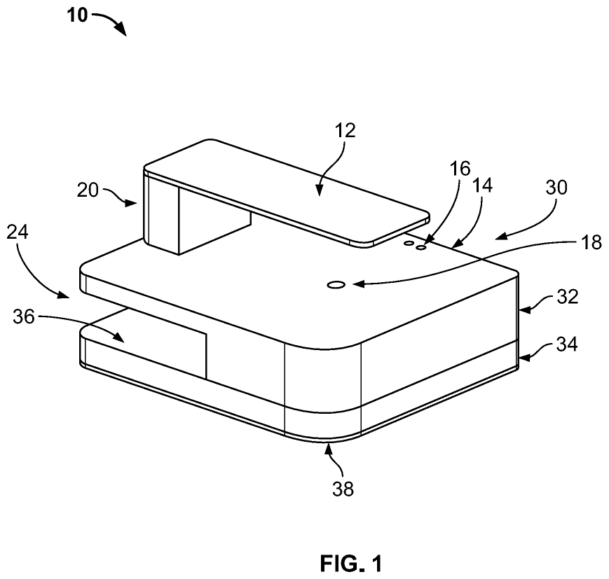

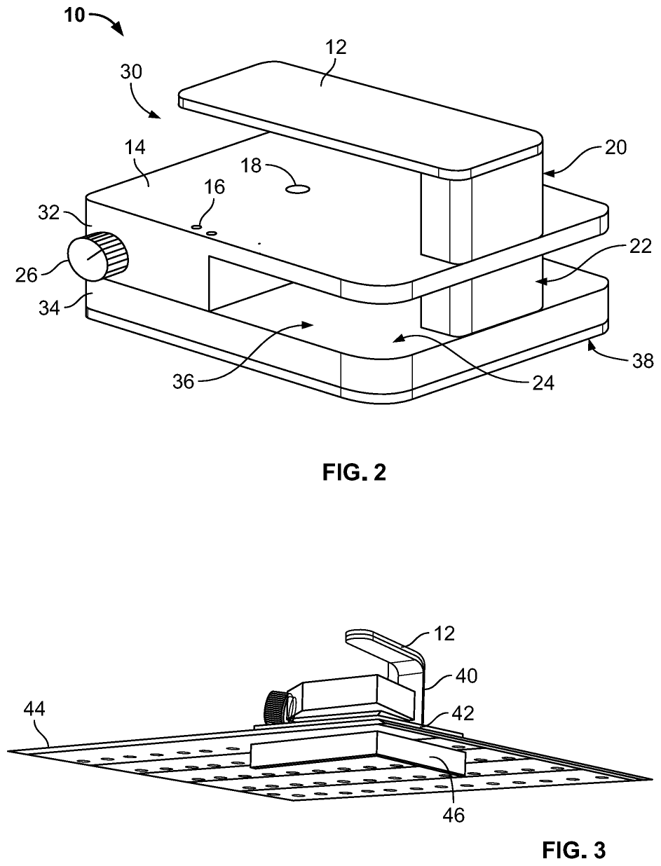

FIGS. 1 and 2 depict the front and back, respectively, of an embodiment of a microscope 10 according to the present invention. Embodiments may include a stage plate 14, upon which an object to be viewed is can be placed. A circular window 18 for a lens may allow a microscope below to view the underside of the viewing object. A top mast 20 may raise and lower a light holder A, which may hold a light mask slider to provide illumination. Indicator LEDs 16 on stage plate 14 may indicate the status of the microscope.

An embodiment of a microscope may have an upper object area 30, a microscope chamber 32, and a PCB chamber 34. A viewing object may be placed in the upper object area 30, beneath light holder 12 and stage plate 14. Light holder 12 and stage plate 14 may be made of white acrylic. Microscope chamber 32 may have side made of white acrylic, and may have a chamber to hold optical components, and a space 24 to allow heat to dissipate. PCB chamber 34 may include a printed circuit board (PCB), a battery, and other electronic components which may become warm. PCB chamber 34 may have a grey, anodized aluminum PCB cover 36, sides, and base plate 38, which may help with thermal isolation.

As depicted in FIG. 2, in an embodiment, top mast 20 and lower mast 22 may also be made of anodized aluminum, and may raise and lower light holder 12. In an embodiment, a course focus dial 26 on the microscope chamber 32 may be used to manually adjust the course focus.

FIG. 3 depicts an embodiment of a microscope mounted on an incubator shelf. Embodiments of a microscope may include a light holder 12, which rises above an object 40 such as a vessel with a cell culture. In an embodiment, a shelf 44 may have a rectangular cut out to allow a top of the microscope 42 to protrude above the shelf 44, and a bottom portion of microscope 46 to extend below the shelf 44. Screws or other attachment means may mount the microscope to the shelf.

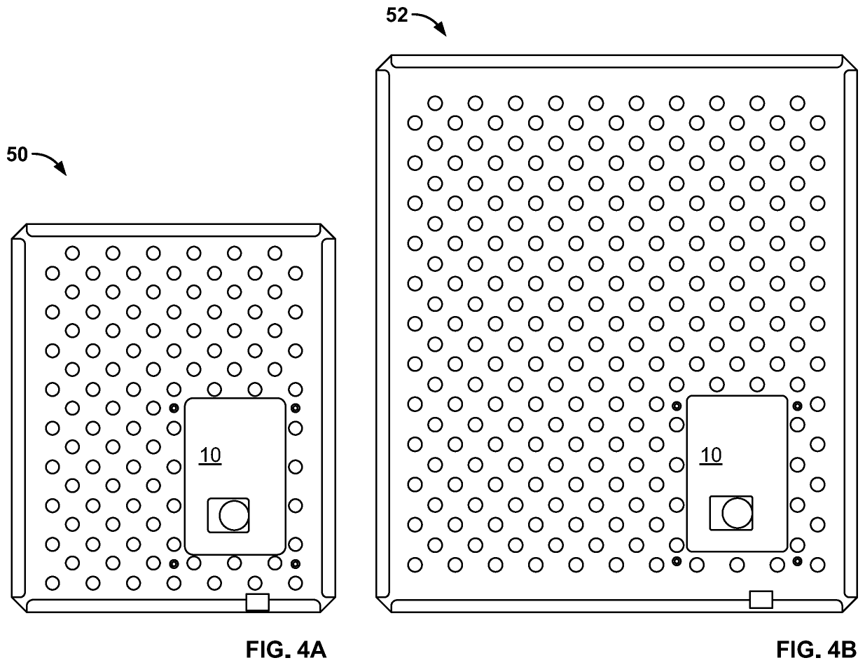

FIGS. 4A and 4B depict, respectively, small-sized 50 and large-sized 52 embodiments of a shelf for an incubator with integrated microscope. Embodiments of a microscope 10 may be mounted near a corner of the shelf, to preserve space on the shelf for other items to be incubated, such as tubes or vessels for cells to be cultured.

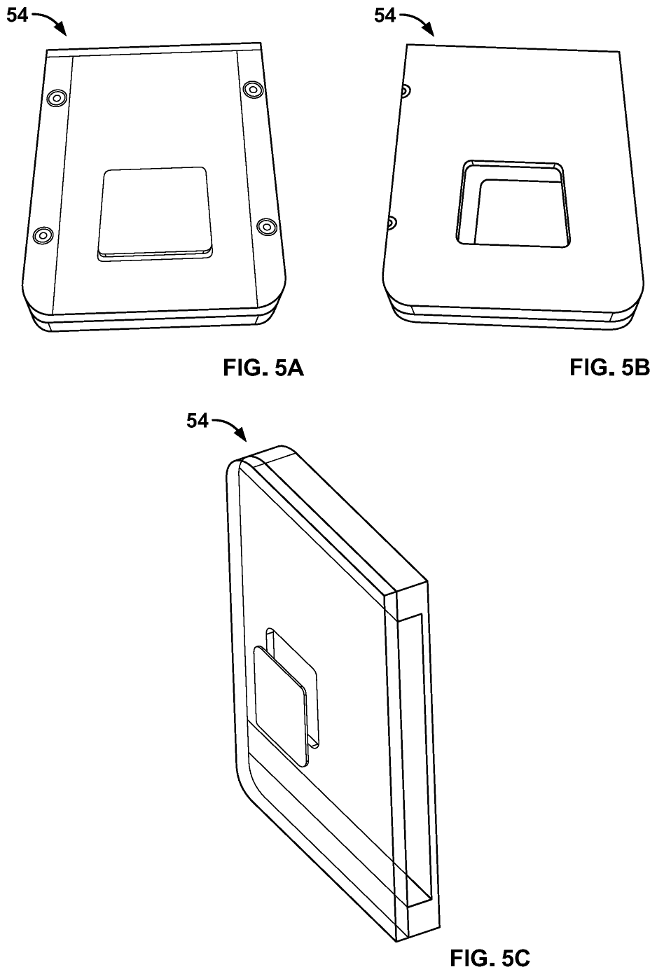

FIGS. 5A, 5B, and 5C depict an embodiment of a light mask slider 54. FIG. 5A shows a darkfield contrast side. When applied with the black square down, light is deflected and only comes from the sides of the light mask slider. FIG. 5B depicts a brightfield contrast. When applied with the opening-cutout down, light passes through to the object. FIG. 5C depicts a side view of a light mask slider 54.

I claim:

1. A device comprising:
a shelf adapted for use inside an incubator;
a microscope integrated into the shelf; and
a wireless transmitter that wirelessly transmits an image produced by the microscope.

2. The device of claim 1, wherein the device is adapted to receive power from an incubator.

3. The device of claim 1, wherein the wireless transmitter further receives wireless signals to operate the microscope, including fine focus.

4. The device of claim 1, further comprising a display; wherein the wireless transmitter transmits the image to the display.

5. The device of claim 1, further comprising controls that provide wireless signals to operate the microscope.

6. The device of claim 1, further comprising:
an incubator; and
a display on an outside surface of the incubator;
wherein the wireless transmitter transmits the image from inside the incubator to the display.

7. The device of claim 1, further comprising:
a stage plate adapted to support an object to be microscopically viewed by the microscope.

8. The device of claim 7, further comprising:
a clear window in the stage plate; and
an objective lens beneath the clear window;
wherein the microscope views the object through the clear window from below the object.

9. The device of claim 7, further comprising:
a light holder attached to a top mast, which raises and lowers the light holder relative to the stage plate.

10. The device of claim 1, further comprising:
a light mask that slides onto a light holder in either of two orientations, which allows for differing illumination conditions

11. The device of claim 1, further comprising:
a printed circuit board (PCB) for the microscope and the wireless transmitter; and
a PCB housing that provides thermal protection from heat generated by the PCB, microscope, and wireless transmitter.

12. The device of claim 11, further comprising:
a space between the PCB housing and the stage plate that heat to dissipate.

13. The device of claim 1, further comprising:
a control to adjust course focus of the microscope.

14. A device for use with an incubator and a display outside the incubator, comprising:
a shelf adapted for use inside the incubator and adapted to receive power from the incubator;
a microscope integrated into the shelf that produces a magnified image of an object inside the incubataor; and
a wireless transmitter that wirelessly transmits the magnified image to the display.

15. The device of claim 14, further comprising:
a stage plate adapted to support the object;
a light holder attached to a top mast, which raises and lowers the light holder relative to the stage plate;
a light mask that slides onto the light holder in either of two orientations, which allows for differing illumination conditions;
a clear window in the stage plate;
an objective lens beneath the clear window to view the object through the clear window;
a printed circuit board (PCB) for the microscope and the wireless transmitter;
a PCB housing that provides thermal protection from heat generated by the PCB, microscope, and wireless transmitter;
a space between the PCB housing and the stage plate that heat to dissipate; and
a control to adjust course focus of the microscope.

16. A device comprising:
an incubator;
a display outside the incubator;
a shelf adapted for use inside the incubator and adapted to receive power from the incubator;
a microscope integrated into the shelf that produces a magnified image of an object inside the incubataor; and
a wireless transmitter that wirelessly transmits the magnified image to the display.
